(19) United States
(12) Patent Application Publication
(10) Pub. No.: US 2022/0125627 A1
(43) Pub. Date: Apr. 28, 2022

(54) CONFORMABLE THERMAL THERAPEUTIC DRESSING WITH MEDICAL INTERVENTION AND/OR SENSING CAPABILITIES

(71) Applicant: Stanley A Sansone, Houston, TX (US)

(72) Inventor: Stanley A Sansone, Houston, TX (US)

(21) Appl. No.: 17/490,525

(22) Filed: Sep. 30, 2021

Related U.S. Application Data
(63) Continuation-in-part of application No. 16/255,511, filed on Jan. 23, 2019.
(60) Provisional application No. 63/086,399, filed on Oct. 1, 2020.
(60) Provisional application No. 62/757,251, filed on Nov. 8, 2018.

(51) Int. Cl. A61F 7/03

(52) U.S. Cl. CPC A61F 7/03; A61F 2007/0276

(57) ABSTRACT

Thermal dressings consist of conformable containment pack to be placed on or wrapped around a part of the body providing thermal therapeutics with or without; sensing as a diagnostic tool and/or medical intervention material. Containment pack has an inner layer closest to the skin and an outer layer furthest from the skin; containment pack has at least one volume deflector configure to provide areas of positive and negative curvature when in use; inner layer positive curvature is an outward protrusion for compression directed into skin for splinting and sensor location, and the negative curvature has gap for circuitry and medical intervention material. Volume deflectors are positioned to provide contour matching portions of body covered by said containment pack; and containment pack has a preloaded first material needed to create a thermal reaction.

### FIELD OF THE INVENTION

This invention relates generally to: cooling or heating appliances for medical or therapeutic treatment of the human body including bandages, dressings, absorbent materials, medicinal pharmacological preparations, and first aid kits. An embodiment uses sensors to measure body and wound characteristics that can be displayed in a three dimensional presentation for planning simulation or modelling of surgical operations.

This invention relates generally to wearable thermal devices fillable through at least one port. Wearable thermal devices are used in emergency, through post emergency treatments, and/or may contain sensors used during the medical event.

### BACKGROUND OF THE INVENTION

Placing or attaching a wearable thermal device, an on-demand thermal dressing, or instant cold pack to a body injury is vastly improved by providing functional shaping. Functional shaping provides volume control and fluid flow means, but also provides useful and novel: flex lines for shaping to body parts; areas of compression and coupling; and a composite surface capability when in contact with skin. Composite surface is a surface with more than one type of material is in contact with the skin or body part. Composite surface provides: more precise and detailed areas of compression with parts of the body, like in splinting; sensor positioning; superior sensor coupling; sensor data transmission and circuitry areas; and/or areas for medical interventions. Areas for sensor wiring and medical interventions are generally voids or channels between the thermal dressing and skin, and are generally located around volume deflectors. Medical interventions include but not limited to: facilitate absorbance or draining of excessive fluid loss in wounds; and/or introduction of pharmacological materials.

Most thermal or therapeutic treatments applied to the human body tend to be thermal packs, heating pads, ice packs, instant cold packs, cooling blankets or electric blankets. These other treatments are intended to lay on a body part though straps or other fastening systems or mechanisms have been devised to hold these thermal treating devices to the body, but lack novel elements of this invention.

### SUMMARY OF INVENTION

This invention provides a conformable chemical activated thermal therapeutic dressing that conforms to or wraps around a part of the body. One embodiment is a cooling therapeutic dressing with splint functionality.

This invention relates to conformable thermal therapeutic dressing with sensors to provide thermal therapeutic treatment and diagnostic tools. Various sensors and sensor types are deployed using a conformable containment pack that provides coupling, transmission, and circuitry. Sensors measure for assessment purposes, including but not limited to: tissue damage; blood flow characteristics; oxygen saturation; and/or temperature. Spatially rendered measurements from located sensors of wound characteristics can be displayed in three dimensional graphical representations as a diagnostic tool for; wound monitoring without removing dressing, and/or planning simulation or modelling of surgical operations.

This invention relates to thermal therapeutic dressing with medical intervention capabilities including: introduction of pharmacological like antibiotics, pain relief, and medicines; application of biomolecule materials like collagen or keratin material; and wound management including control fluid loss with absorbance material.

This invention provides a controlled heating patch with pharmacological material attached to enhance transdermal transfer, including pain relief, vaccines, and biomaterials/biomolecule materials.

This invention relates to an apparatus that can provide therapeutic thermal treatment to a part of the body and may be filled by end user or customer at a selected time. An example is a wearable thermal device of a selected shape and selected volume is shipped to a customer without thermally treatable material. Advantages include: less shipping weight; less shipping size and packaging; less shipping costs; less packaging costs; less environmental costs, both direct and indirect; providing the customer ability to refresh contents of wearable device thereby decreasing waste. A novel feature is: said selected volume is use to provide a selected amount of thermal energy; and/or selected volume is controlled by selectively sealed portions of device which also functions to control flow and pooling.

This invention relates to an apparatus to deliver thermal therapeutic treatment that: wraps around a part of the body for thermal treatment; completely covers an appendage; and/or, completely cover a body. Such wraps may include, but not limited to: appendage; neck; or thorax. Wearable thermal device may act like a bag with or without a long sleeve to completely cover an appendage. Wearable thermal device may be a body bag to control a desired temperature of a whole body.

This invention relates generally to an apparatus port to assist in the filling of a wearable thermal device. An advantage is the wearable thermal device may be filled and refilled by the customer or purchaser of the product and not by the manufacturer. Advantage is reduction of shipping and packaging costs, and labor.

An important characteristic of the present invention is the immediate application of thermal treatments from a wearable thermal device in which a chemical reaction is initiated when at least another and necessary material is separately added through port at a selected time. It is well known that the quick application of selected thermal treatments can be a therapeutic and life-saving during a medical event. This eliminates the extra weight and size of wearable thermal device when second component is not carried in or on wearable thermal device.

An advantage is the wearable device may be safer to carry. Since the second and needed component or material is not present in the system, accidental activation of thermal chemical reaction and the release of thermal energy are eliminated. For example, a thermal device like an instant cold pack may contain a separate package of water. Eliminating the water from the device reduces its accidental activation and subsequent undesirable timing of an endothermic reaction.

This invention relates generally to an apparatus for the immediate application of cooling treatments to different parts of an injured body. It is well known that the quick application of selected hypothermic treatments can be a therapeutic and life-saving event. Quick application can reduce the effects of an injury and in many instances: arrest the injury from worsening; decreases cellular permeability; and vasoconstriction. An important characteristic of the present invention is that a chemical reaction within wearable thermal device is the most immediate and targeted application possible. Surgical facilities already overloaded with equipment would not need equipment to cool a patients appendage or body part as this function is self-contained within wearable thermal device. Even if additional cooling is required a much smaller cooling unit within the surgical unit would be a great advantage over the prior art.

This invention relates generally to an apparatus for the quick application of warming or hot treatments to different parts of an injured body. The timely application of form fitting warming therapy compress device reduces the effects of hypothermic injury, aids in the healing process after surgery, and promotes blood flow and vasodilation. Chemical reaction within an apparatus to apply thermal treatments generally is the fastest and quickest application possible and is an important characteristic of the present invention.

Another advantage is multiuse port may be, or consist of, at least one valve. The use of at least one valve also allows for: the. At least one valve may be used to: control pressure within wearable thermal device; introduce into wearable thermal device chemical or material needed to activate chemical thermal reaction; introduce thermally treatable material; introduce thermally treated material; and/or minimize leakage.

An advantage of invention is to provide a multiuse port where 2 types of thermally treatable material introduction are provided; non-pressure and pressure. Multiuse port may be simply used to allow the pouring or introduction of thermally treatable material into wearable thermal device, described as non-pressured introduction. Same multiuse port may consist of at least one valve where introduction of thermally treatable material may be injected into wearable thermal device, described as pressured introduction. A multiuse port provides optional usability for different situations. These different situations include: tactical combat zone; emergency scenes; and/or surgical setting. Having a single wearable thermal device used throughout various medical situations offers advantages including but not limited to: saves manufacturing costs; save lives; minimizes training; and other advantages.

This invention relates generally to an apparatus comprising a port with at least 2 valves to extend thermal treatment. Valves allow fluids or gases to circulate throughout wearable thermal device. A particular embodiment of invention relates an apparatus for the extended and longevity of thermal treatment. For example, when an endothermic derived cooling treatment expires or warms the valves of the port may be connected to external cool treating machine where material is thermally conditioned and then circulated through port and into wearable thermal device. Thermally treated materials generally refers to liquids and/or gases that are treated, conditioned, heated and/or cooled externally and then introduced into wearable thermal device. This creates a novel apparatus that can be activated to deliver thermal treatment within seconds and then last indefinitely.

Another advantage is to provide a multiuse port with at least one screen or filter to control flow. Control may mean limiting contaminants from being transported into wearable device as in a filter, where contaminants may be: material that can puncture wearable device; improper material; and/or material that can cause blockage within wearable device. Control may mean to protect valves from blockage that may get blocked from contaminants and/or unspent materials.

Another novel feature is to provide screens and/or filters that may be used to provide uniform distribution and/or application of contents expelled from wearable thermal device or containment pack. This novel feature provides a second use capability for wearable thermal device. When contents such as those from a used up chemical reaction of ammonia nitrate and water can be expelled or projected from wearable thermal device said screen can aid the novel distribution of contents onto the ground for the purpose to fertilize fields and grow food.

Another advantage is to provide barriers to distribute desired thermal treatments evenly throughout the said wearable device. Barriers or volume deflectors have been used to control the flow of externally thermal treated material throughout the wearable thermal device. A novel feature is the use of a semi-permeable volume deflectors where instead of a single barrier or fence line said semi-permeable volume deflectors can be made of small sealed portions of containment pack organized into lines and provide novel flow to eliminate blockage in different portions of wearable device. Barriers or volume deflectors are also used to: control volume, a selected volume, or a selected amount of volume in a novel way. Volume can be used to determine the amount of thermal mass energy that can be transferred to reach desired target temperatures of appendage or body part; and/or create compartments for pooling or targeting of thermal energy.

Another novel feature of invention is ability to control temperature and amount of heat to be transferred. Control of temperatures may consist of selecting an amount of each component needed to create a certain amount of thermal energy created by a thermal reaction. The amount of heat to be transferred is controlled by selecting a volume of thermal energy consistent with target temperatures.

It is the intent of the current invention to provide onboard sensors. An advantage of this novel feature is the coupling application. A novel feature is the superior and consistent coupling of biosensors, and/or providing body part or body diagnostics during thermal therapeutic treatment. Onboard sensors are connected or attached to wearable thermal device and/or containment pack, and may include, but not limited to: device sensors to measure system or device, performance or diagnostics; and/or biosensors to measure physical and biological characteristics of appendage or body part covered by wearable thermal device. Onboard sensors may be placed in a separate layer, called a sensor layer. Examples include but not limited to: temperature sensors for the device connected and unconnected; sensors to measure temperature of appendage and/or body part; electrical sensors as in EEG electroencephalogram; and or optical sensors as in PPG, photoplethysmography. An example is a wearable device consisting of a single layer of multiple PPG sensors with the intent to measure in a 3 dimensional (3D) analysis: blood flow; blood pressure; cellular pressure; and/or oxygen levels of appendage or body part. Other advantages include, but not limited to: creation of a 3D volume; images; and/or video for detailed observation of appendage or body part function.

This invention relates generally to an apparatus which uses biosensors to control thermal regulation. Such control may include, but not limited to: biosensors communicate with at least one valve with aperture or valve control; biosensors may communicate through port junction to external thermal treating machine, where said machine may control temperature and/or pressure flow; and/or some combination.

Another advantage is to provide a port consisting of 2 valves of different sizes and/or flow characteristics. Different valves sizes may maintain and/or control a selected pressure within or inflatedness of wearable device. For example, the flow of fluids and/or gases: into a wearable device from an inlet valve which is larger and/or greater than; flow out of the wearable device through an outlet valve which is smaller than inlet valve. Inflatedness of wearable thermal device can be controlled by external machine or special sealing of volume deflectors around out take valves.

### BRIEF DESCRIPTION OF THE DRAWINGS

FIG. 1 shows a wearable thermal device with port and sensor.

FIG. 2 shows a foot wearable thermal device with port.

FIG. 3A shows an appendage wearable thermal device configured as a bag.

FIG. 3B shows a cross section of appendage wearable thermal device of FIG. 3A.

FIG. 4 shows multiuse port assembly.

FIG. 5 shows cross sectional view of multiuse port body.

FIG. 6 shows a top view into body of multiuse port body.

FIG. 7 shows multiuse port assembly.

FIG. 8 shows multiuse port in combination with a foot wearable thermal device.

FIG. 9 shows multiuse port in combination with a head wearable thermal device.

FIG. 10 shows wearable thermal device with biosensors.

FIG. 11 shows conformable compressive therapeutic dressing with volume deflectors.

FIG. 12 shows three cross sections (A-C) through conformable compressive thermal therapeutic dressing of FIG. 11.

FIG. 13 shows a wearable conformable thermal therapeutic dressing for treatment of the face and neck.

FIG. 14 shows an instant thermal conformable therapeutic dressing.

FIG. 15 shows a top view of a thermal bandage.

FIG. 16 shows a cross section D through thermal bandage of FIG. 15.

FIG. 17 shows the pattern of molded composite surface.

FIG. 18 shows a transport pattern of molded composite surface.

### DETAILED DESCRIPTION OF THE PREFERRED EMBODIMENTS

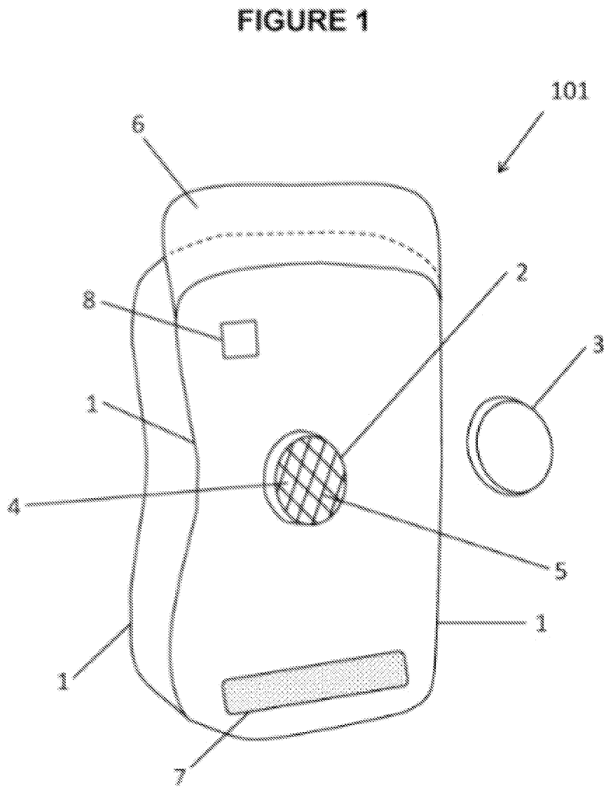

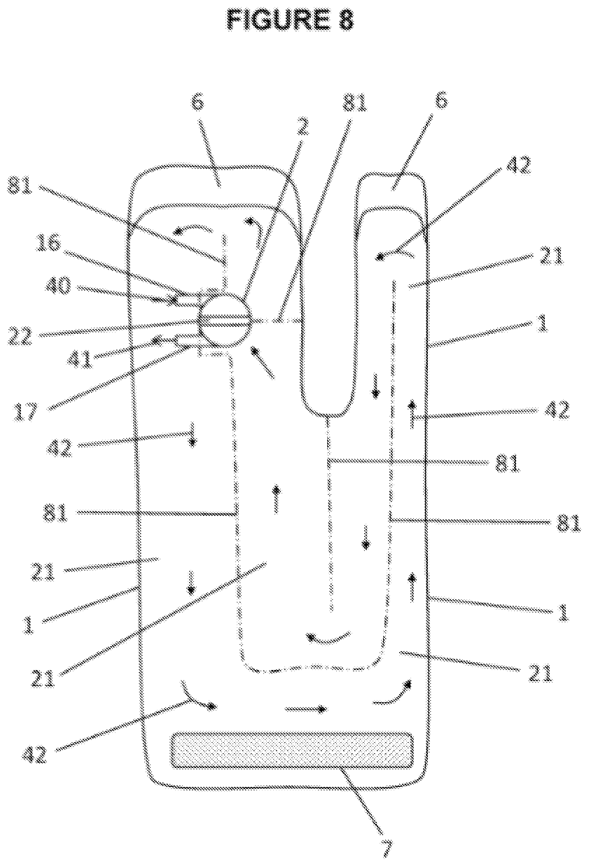

FIG. 1 shows a wearable thermal device 101 consisting of containment pack 1 and port body 2. Port body 2 is attached to containment pack 1 and provides port opening 4 into volume 21 (see FIG. 8, 9). Containment pack 1 may be, including but not limited to: the wearable thermal device; have layers attached such as insulation, skin protection, and/or barriers; and/or a bag or bladder which may be inserted into said wearable thermal device. Containment pack 1 consists of: selected shape; selected internal volume; and/or selected internal configuration. Containment pack 1 is fillable with thermally treatable material. Screen 5 may be used for functions such as, but not limited to: provides a filter to limit objects from entering and harming the containment pack 1; limits spillage of preloaded component out of containment pack 1; protect components of multiuse port; and/or provides a selected projection of contents from within containment pack 1 similar to a shower head. Screen 5 may be curved or channeled for a desired application of projection when contents are expelled from wearable thermal device and/or containment pack 1. Closing cap 3 seals contents of containment pack 1 to prevent leakage. Fastening system may consist of fastening flap 6 and fastening counterpart 7. Fastening flap 6 and fastening counterpart 7 allow wearable thermal device to: stay wrapped around a limb, spine, thorax or other body part; and/or hold appendage wearable thermal device in place. Fastening flap 6 and fastening counterpart 7 may consist of: zipper; hook and loop; or other fastening system. At least one sensor 8 is attached to containment pack 1 and may consist of at least one temperature sensor or other sensors. Temperature sensors may function to measure: temperature of materials within containment pack 1; and/or temperature of body part covered by wearable thermal device. Sensors may be used to: body part monitoring and measurement; imaging; determine treatment and/or adjust treatment; and/or provide feedback for autonomous regulation of treatment.

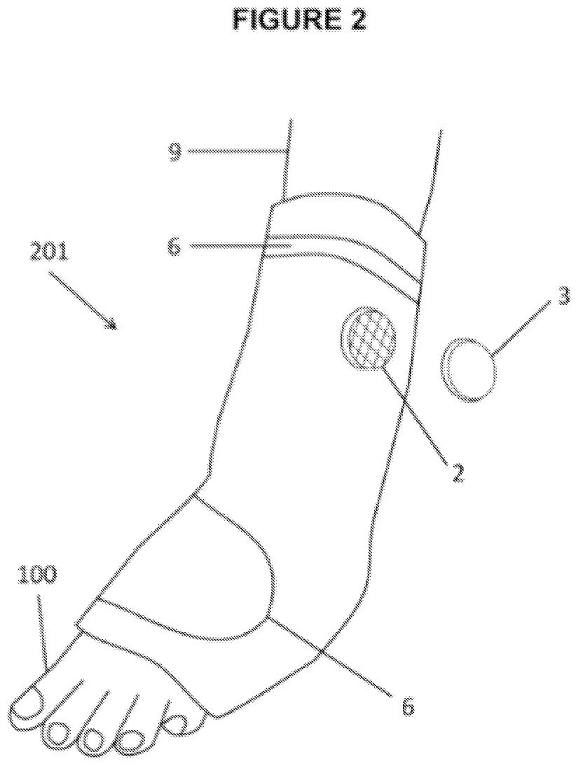

FIG. 2 shows a wrapping type foot wearable thermal device 201 with port body 2 and closing cap 3. Containment pack 1 wraps around a selected portion of a leg 9 and to the toes 10. Fastening flap 6 and fastening device 66 may have closing counterparts attached to containment pack 1, and/or a cord and lock mechanism to secure wearable thermal device around a foot and selected length of leg 9.

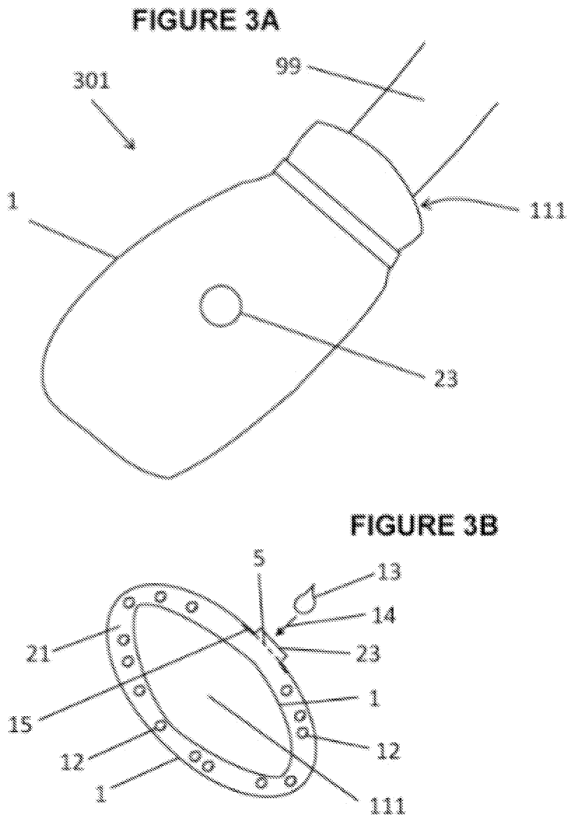

FIG. 3A shows a bag type appendage wearable thermal device 301. An appendage for which the bag type appendage wearable thermal device may be deployed include: hand; hand and arm 99; foot; foot and leg 9; or head. Said bag type appendage wearable thermal device can be similar to a tube with one end closed. The open end of closed tube or bag is appendage insertion opening 111 where an appendage is inserted. A mitten or bag would best describe said bag type appendage wearable thermal device 301 to cover hand. Another type of appendage wearable thermal device may be mitten or bag that includes a section or sleeve going up a selected length of arm 99. Likewise a sock may best describe said bag type appendage wearable thermal device to cover foot. Sock includes a section or sleeve going up and covering a selected length of leg 9. Port assembly 23 is attached to containment pack 1. Port assembly 23 allows for the introduction, storing, and/or sealing of thermally treatable material to be placed into said containment pack 1. Said thermally treatable material may include, but not limited to: water; gel; water and additives such as alcohol, ethanol and others, salt water; rice; oxygen, air, and/or other gases; and/or a thermally reactive material. Fastening device 66 may be hook and loop, and/or a cord and lock mechanism.

FIG. 3B shows a cross section through bag type appendage wearable thermal device 301 of FIG. 3A. Containment pack 1 has appendage insertion opening 111 to receive appendage. Within said containment pack 1 is a selected amount of preloaded component 12. Preloaded component 12 is at least one chemical material needed to initiate or activate a chemical thermal reaction. A thermally treatable material which is a second and needed chemical to initiate or activate said chemical thermal reaction is called introduced component 13. A selected volume of introduced component 13 may be poured through port assembly 23 and/or injected into said containment pack 1 at a selected or desired time. Arrow 14 shows direction of introduced component 13 into containment pack 1. Said preloaded component 12 and introduced component 13 when combined together produce a selected amount of chemical thermal reaction. Said selected chemical thermal reaction may be an endothermic or an exothermic reaction for the purpose of providing: immediate thermal therapy; and/or a measured amount of thermal therapy such as cooling therapy or heating therapy, respectively. An example of an endothermic reaction is: a selected amount of ammonia nitrate is preplaced in containment pack 1 and is called preloaded component 12; then at a selected time, a selected amount of water called introduced component 13 is poured or injected through port assembly 23 into containment pack 1; and when preloaded component 12 and introduced component 13 mix create a selected amount of thermal energy or cooling effect for therapy. Screen 5 may be used, in part, to keep preloaded component 12 from falling out of containment pack 1. An example of an exothermic reaction is iron powder as preloaded component 12 and introduced component 13 may be oxygen or air.

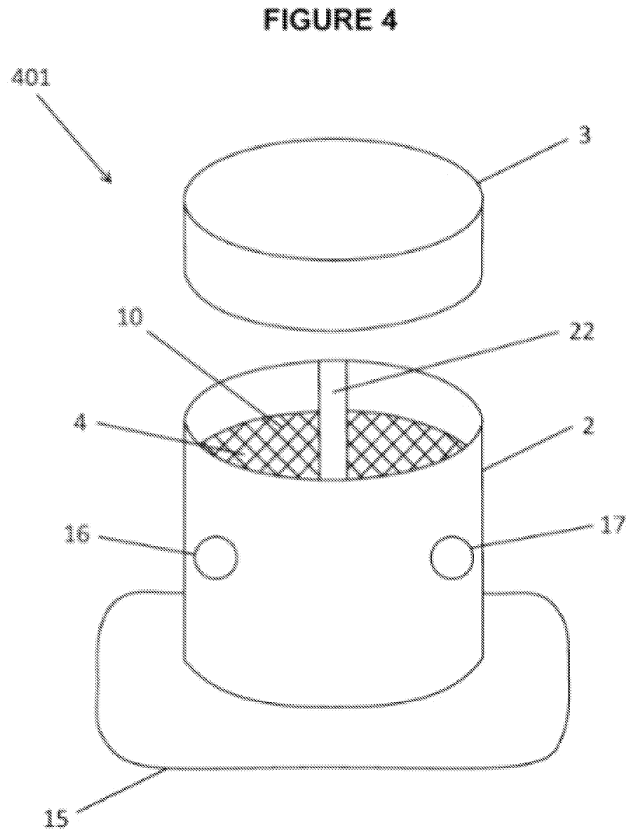

FIG. 4 shows an example of a multiuse port assembly 401. Port body 2 has multiple functions that include, but not limited to: thermally treatable material may be poured into containment pack 1 through port body 2 and port opening 4; and/or thermally treatable material may be introduced into containment pack 1 and port opening 4 through at least one valve 16, 17 where said thermally treatable material is under pressure. Flange 15 is used to join port 2 to containment pack 1 (also see FIG. 3B). Flange 15 may be separately sealed to containment pack with a fitting that would allow port body 2 to be placed. Port divider or port partition 22 divides port body 2 and port opening 4. Both sides of port body 2 and port opening 4 open into volume 21 and at different ends of containment pack 1. Valves 16, 17 are shown to partially protrude into port opening 4 in order to: best describe; and/or provide stronger port structure. Said valves 16, 17 may not protrude into port opening 4, in order to provide larger or a greater port opening 4. Valves 16, 17 may be, but not limited to: drip-less values; leak-less values; pneumatic valves; compression coupling; and/or twist to connect values. A selected version is where port body 2 is permanently sealed, or cap 3 is not necessary as it becomes part of port body 2 with access only through at least one valve. Upper screen 10 may be used to protect valves

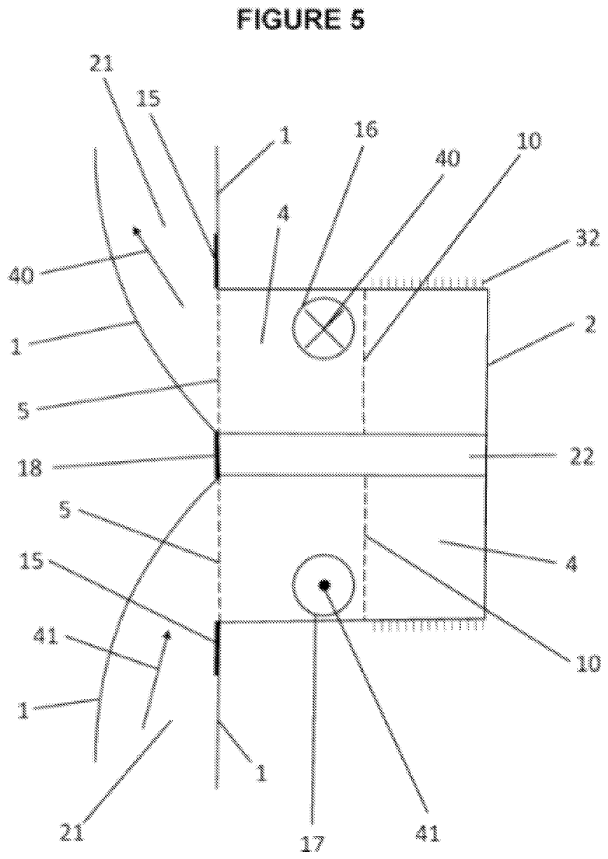

FIG. 5 shows cross sectional view of port body 2 used in multiuse port assembly 401 (see FIG. 4). Port body 2 is sealed or attached to containment pack 1 by: flange 15; and port partition seam 18. Port divider or port partition 22 is sealed to containment pack 1 along port partition seam 18. Valves 16, 17 function to introduce and/or removal of contents, into and out of containment pack 1. Port divider or port partition 22 separates flows going in different or opposite directions; and generally located at each end of containment pack 1 (see FIG. 8, 9). Screen 5 and upper screen 10: protect valves from blockage; prevent debris from entering containment pack 1; and may also be used to project contents of containment pack in a controlled and/or distributed manner. Projection of contents can be achieved by simply squeezing containment pack 1 or wearable thermal device resulting in a spray of mixed and thermally spent preloaded component 12 and introduced component 13 (see FIG. 3B). For example containment pack 1 may contain a mixture of ammonia nitrate and water which is a fertilizer. Novel use of said screen 5 and/or upper screen 10 is to control projection or spray out onto a patch of land much like a shower head for the intent to grow food, thus providing a second use for wearable thermal device. Intake valve 16 may provide for the pressured introduction of: external fluids or gases; introduced component 13 into containment pack 1 to activate desired thermal reactions; and/or an inlet for circulating externally thermally treated material. Valve 17 is use as out take of material in containment pack 1 and in combination with valve 16 to function together to extend thermal treatments by circulating externally thermally treated fluid and/or gas from an external source through containment pack 1 (see Welkins, U.S. Pat. No. 6,551,347). For example, flow into containment pack 1 and/or volume 21 through intake valve 16 is intake valve flow direction 40. Flow out of containment pack 1 and/or volume 21 through outtake valve 17 is outtake valve flow direction 41. Material flow can be controlled by: intake valve 16 is larger than out take valve 17; external thermal machine; and/or flow restrictions using volume deflectors 81 close to out take valve 17. Closing cap 3 goes over port body 2 to close or seal off port opening 4 with a closing mechanism that may be, but not limited to: screw type; quick release coupling; twist and connect coupling; some other sealing mechanism; and/or permanently sealed. Screw type threaded mechanism 32 as in a male screw type is shown and said closing cap having complementary female type treading.

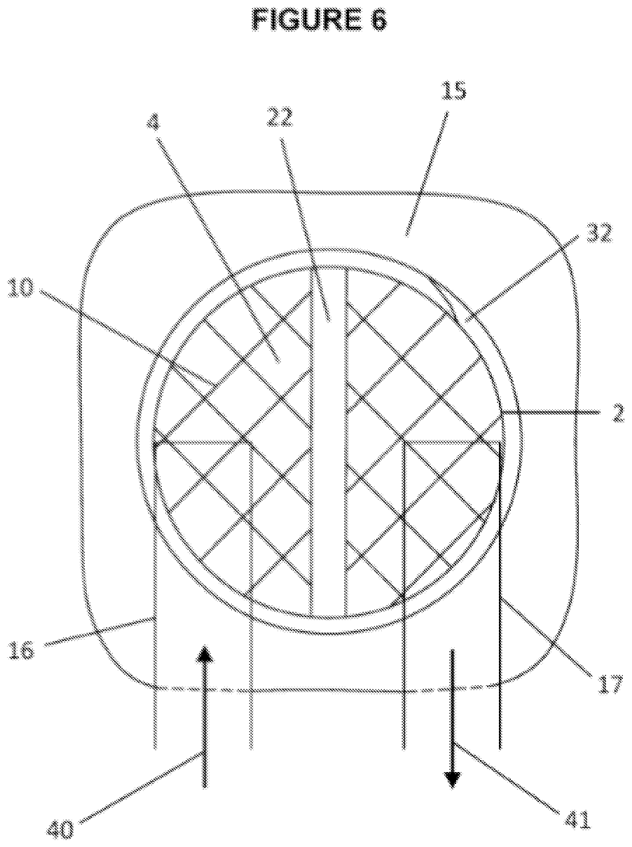

FIG. 6 shows a top view into body of multiuse port body 2 used in multiuse port system 401 (see FIG. 4). Flange 15 is used to seal port body 2 to containment pack 1 and/or wearable thermal device. Closing mechanism or male screw threaded mechanism 32 is used in conjunction with a female screw type closing cap and seals port opening 4. Port divider or partition 22 functions to separate: different ends of containment pack 1; different ends of volume 21; valves 16, 17; and different flow directions 40, 41. Valves 16, 17 may be connected, by hoses, to external source of thermally treated material to extend thermal treatment.

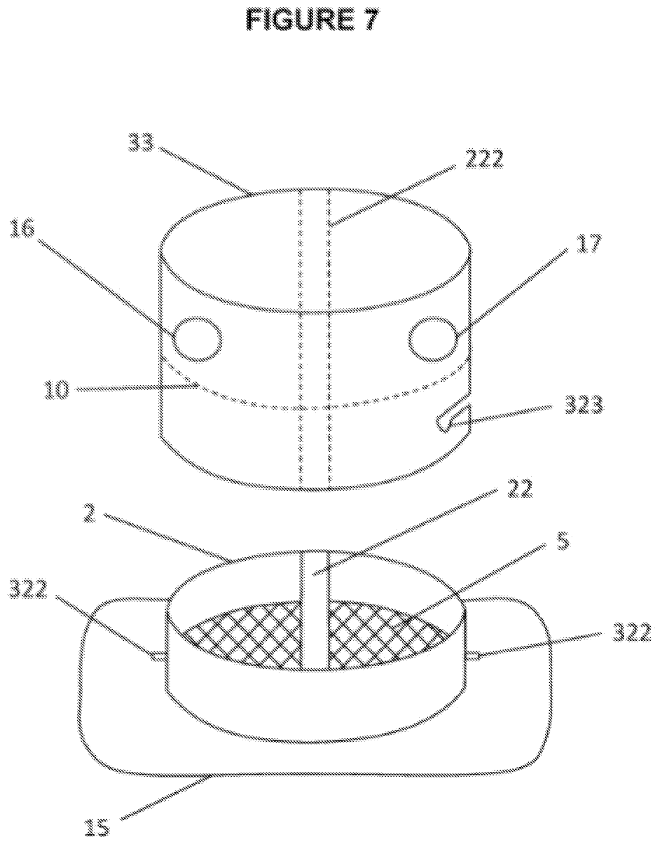

FIG. 7 shows a particular embodiment where some functionality of multiuse port assembly is positioned in port body 2, and others are positioned in closing cap 33. Port body 2 is attached to containment pack 1 with flange 15 and port partition seam 18. Port body 2 may include, but not limited to: port divider or port partition 22; screen 5; and/or port body closing mechanism 322. This configuration of port body 2 allows for the easy and accessible pouring of thermally treatable material into containment pack 1 and volume 21. Valve closing cap 33 consists of at least one valve and contains other functionality of multiuse port. Valve closing cap 33 may include, but limited to: port cap divider or partition 222; upper screen 10; intake valve 16; out take valve 17; and/or cap closing mechanism 323. Port cap divider or partition 222 and upper screen 10 are located within the valve closing cap 33 and because they are hidden from this view they are represented by dashed lines. Port cap divider or partition 222 is intended to: align with port divider or port partition 22 of port body 2; seal both ends of containment pack 1 when attached to port body; and/or separate different flow directions into and out of containment pack 1. At least one cap closing mechanisms 323 may be a press and twist to connect type connector which is intended to mate with at least one port body closing mechanism 322. Normally two closing cap closing mechanism 323 and two port body closing mechanism 322, but there may be more such as three to better secure valve closing cap 33 to port body 2. Upper screen 10 is inside valve closing cap 33 and is therefore represented by dashed lines. Upper screen 10 protects valves from debris associated with objects not passed through screen 5 and remaining in port body 2. Multiuse port functions to allow for the introduction of a selected amount of thermally treatable material and/or thermally treated material through at least one valve. Valves 16, 17 are integrated into valve closing cap 33 assembly and act in the same manner as described above with different flow directions 40, 41

FIG. 8 shows multiuse port in combination with a wrapping type foot wearable thermal device (see FIG. 2) which is intended to wrap around foot and selected portion of leg 9. Multiuse port body 2 is shown with intake valve 16, out take valve 17 and port divider or port partition 22. Intake valve 16 provides intake valve flow direction 40. Out take valve 17 provides for out take flow direction 41. Port partition seam 18 and volume deflectors 81 further assists in separating each end of containment pack 1. Volume deflectors 81 may consist of: sealed lines within outline or selected shape edges of said containment pack 1; selectively sealed lines of limited permeability within outline or selected shape edges of said containment pack 1; and/or separate barriers selectively placed to separate compartments and/or flow channels. Volume deflectors 81: control flow and flow direction 42 between valves 16, 17, where flow direction 42 is indicated by arrows; create thermal compartments, and/or flow channels within containment pack 1; may provide for selected distribution of preloaded component 12 within containment pack 1 (see FIG. 3B); provide control of selected amount of volume 21; and/or control amount of introduced component 13. A particular embodiment of this invention features initial temperature controls and/or thermal mass control. Initial temperature controls and/or thermal mass transfer capability control can be realized through varying the amount of said preloaded component 12 and introduced component 13. Selective volume 21 and a selective temperature of material in said selective volume 21 determines a selected amount of thermal energy or thermal mass. Target temperatures needed or desired for thermal therapeutic application of an appendage or body part is used to determine the amount of thermal mass to be created by designing and using a) selected volume 21 and b) selected temperatures. Temperatures are controlled by specifying the: amount of thermal reaction; amount of preloaded component 12; and/or amount of introduced component 13. Each fastening flap 6 contains a latching material to attach to fastening counterpart 7. Fastening flap 6 and fastening counterpart 7 allow wrapping type foot wearable thermal device to stay wrapped around a foot and/or leg.

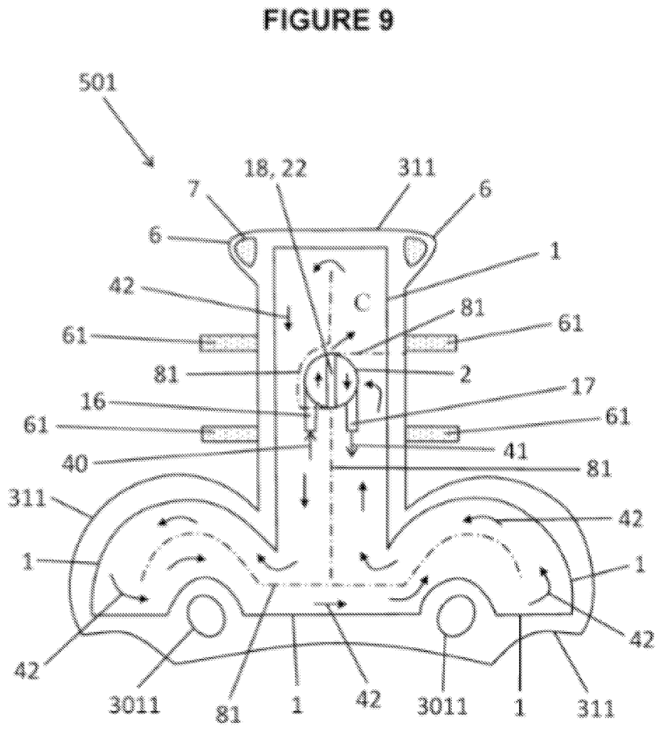

FIG. 9 shows a wrapping, bag type appendage wearable thermal device 501 with one appendage insertion opening that is intended to wrap and close around the head. This particular embodiment comprises a containment pack 1 within wearable thermal device 301. Appendage wearable thermal device 501 has a selected wearable thermal device outline 311 and contains containment pack 1. Containment pack 1 may be inserted into and/or attached to appendage wearable thermal device 501 and may have layers. Layers are functional and include, but not limited to: thermal insulation; radiant barrier; sensors; and/or skin protectant. Said appendage wearable thermal device uses fastening mechanism strap 61 and fastening counterpart 7, which may be hook and loop, or other fastening system to close appendage wearable thermal device 501 around a head. FIG. 9 intended view is from the inside of wearable thermal device hence fastening counterpart 7 associated with fastening mechanism strap 61 are not shown. Port body 2 of multiuse port is shown with intake valve 16, out take valve 17, and port divider or port partition 22. Port divider or port partition 22 is located in the same position as port partition seam 18, in this view. Valve 16, 17 provide access to both ends of containment pack 1 and are separated by: port partition seam 18; port divider or port partition 22; and volume deflectors 81. Intake valve 16 provides intake valve flow direction 40. Intake valve 16 can be used to: fill containment pack 1 with thermally treatable material; fill containment pack 1 with introduced material 13; and/or add external thermally treated fluid and/or gas into containment pack 1 or volume 21. Intake valve 16 may be used to transport introduced component 13 into containment pack 1 or volume 21 to mix with preloaded component 12 (see FIG. 3B) to activate a thermal reaction. Flow direction 42 within containment pack 1 or volume 21 is shown as arrows. Volume deflectors 81, shown as dashed lines, control flow direction 42 to ensure even distribution and coverage of thermal therapeutic treatment. Volume deflectors 81 may also function to control volume 21, create thermal compartments C, and/or shape of containment pack 1. An example of a thermal compartment C within volume 21 is a larger pool of thermal material in the left frontal brain lobe of the wrapping, bag type appendage wearable thermal device 501. Out-take valve 17 may provide out take flow 41 out of said containment pack 1 and/or volume 21. A particular embodiment of current invention is the ability to extend emergency treatment in a medical event through the use of multiuse port and external thermal treating machine. Extended thermal treatments may last for extended periods of time, perhaps for days, weeks or months. Ear cutouts are provided for sound transmission holes 3011.

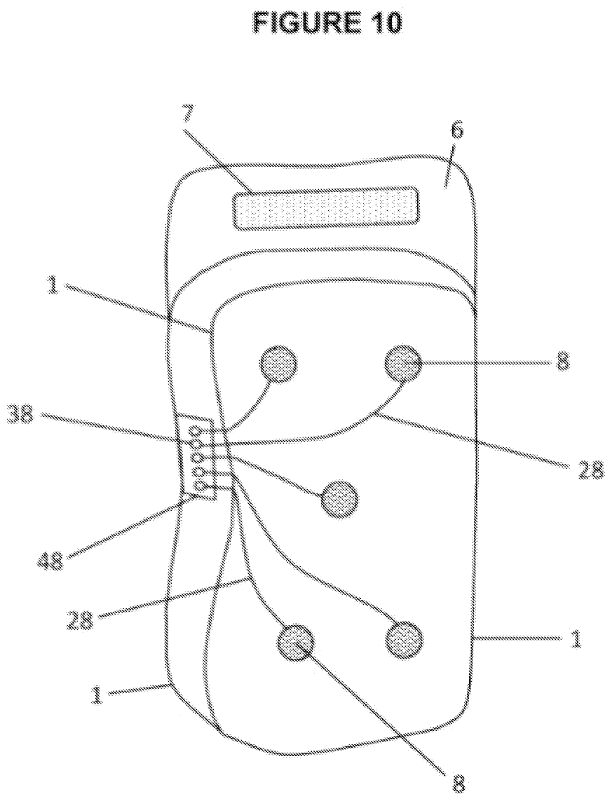

FIG. 10 shows wearable thermal device with biosensors 8. Biosensors 8 that may be used in and/or on wearable thermal device may include: but not limited to: temperature sensors; sensors for body part; electrical sensors as in a EEG, electroencephalogram; and/or, optical sensors as in PPG, photoplethysmography. It is the intent of the current invention to provide a multitude of sensors in a layer to measure appendage or body part physical and biological characteristics. Temperature sensors may be simple temperature strips that display color indications of temperature of device, or connected to a display unit for more accurate reading. Other temperature sensors may monitor body part temperature. Biosensor wires 28 connect biosensors 8 to biosensor port 48 where each sensor has its own connection or channel 38. Note that in FIG. 10, this particular configuration, said port body 2 is on opposite side of containment pack 1 and out of view. Multiuse port may be integrated with biosensor port 48 to provide additional capabilities into multiuse port. An advantage is a multiuse port can now provide patient and device monitoring capability as well as thermal treatments. Biosensor port 48 may include a multiplexer. Said multiplexer may reduce: cost; minimize wire between device and external signal collection unit; and/or reduce weight of device on patient. Fastening flap 6 and fastening counterpart 7 may be a hook or loop material. An example is a wearable device containing multiple PPG sensors with the intent to measure and display a 3 dimensional analysis of; blood flow, blood pressure, cellular pressure, and/or oxygen levels. Biosensors 8 may be gridded in a selected shape, configuration, density and/or pattern to analysis biological characteristics.

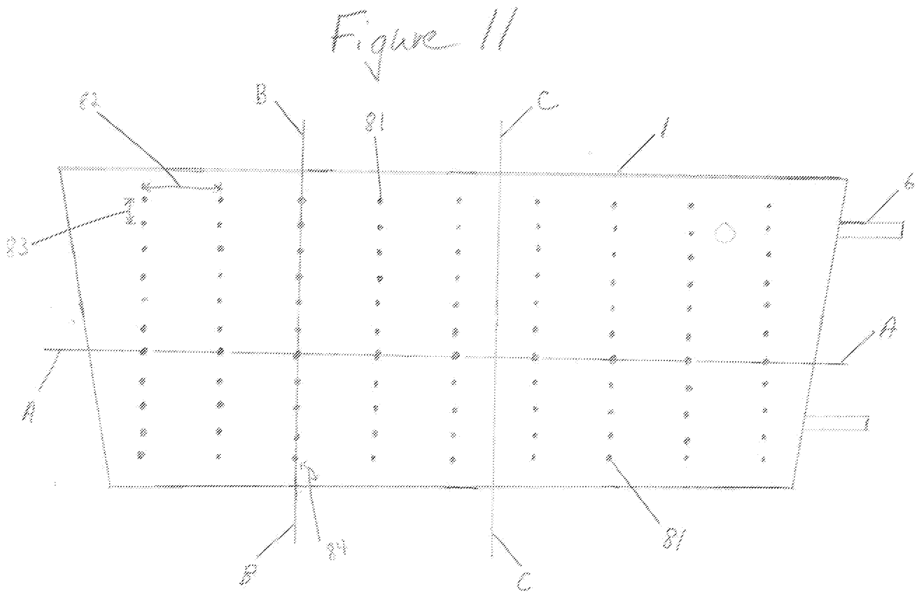

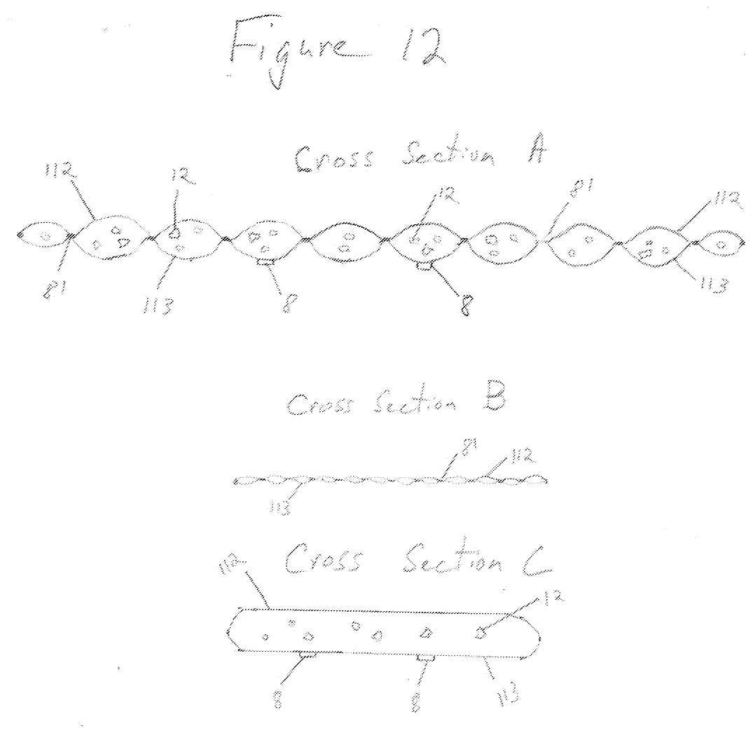

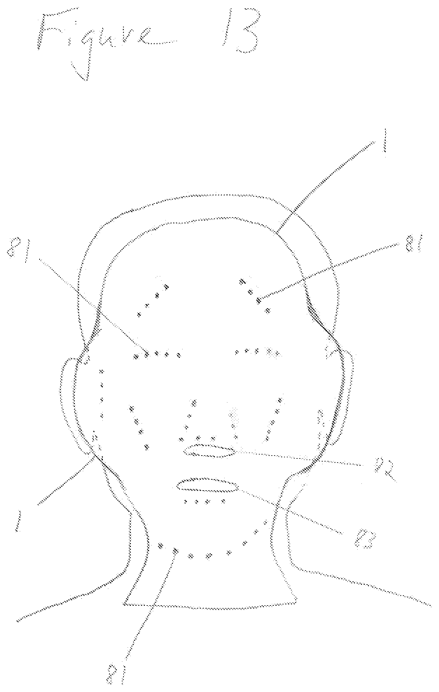

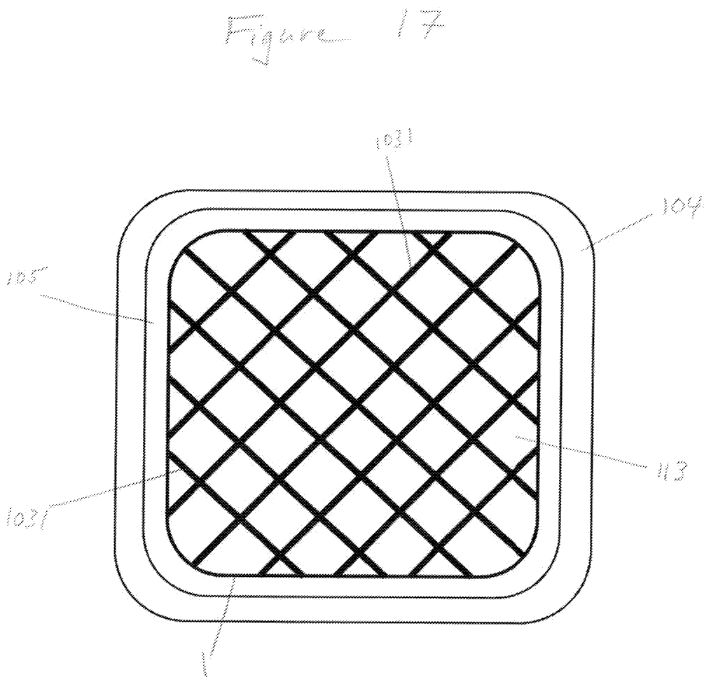

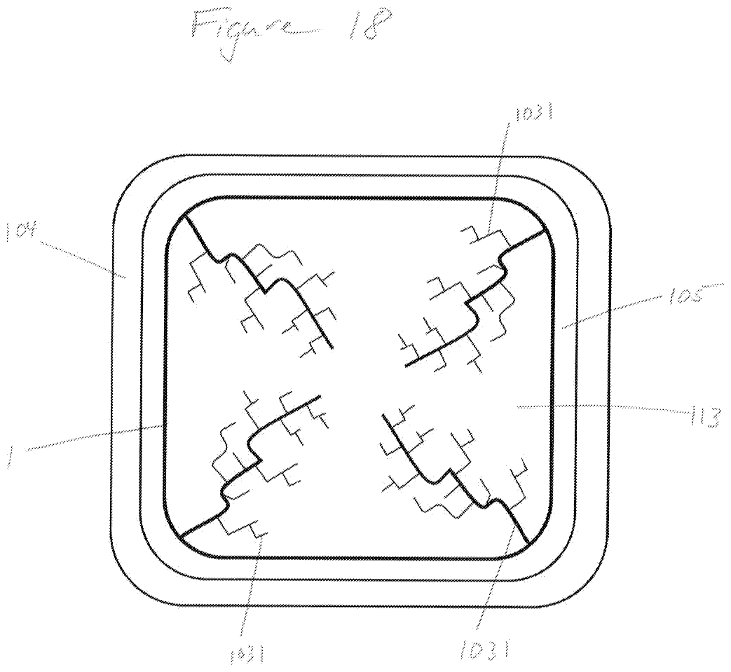

FIG. 11 shows containment pack 1 of conformable compressive thermal therapeutic dressing with volume deflectors 81. Volume deflectors 81 are shown as single circles or dots arranged in lines and a pattern. Lines of volume deflectors 81 provide for flexure or bending to contour to a desired form. FIG. 12 shows three cross sections through conformable compressive therapeutic dressing of FIG. 11 in a state of use or filled. Containment pack 1 has an inner layer 113 proximal to the surface of the skin (closest to the skin) and an outer layer 112 distal to the skin (furthest away from skin). Inner layer 113 and outer layer 112 are selectively sealed at the position of volume deflectors 81 using heat sealing or other sealing methods. Cross Section A is through the long dimension of a filled containment pack 1 of FIG. 11 and through a line of volume deflectors 81. Cross Section B is through the shorter dimension of the containment pack 1 and through a line of volume deflectors 81. Spacing 83 of volume deflectors 81 in Cross Section B provides for a line or lines of flexure or bending. When conformable thermal dressing is activated and placed on or wrapped around a part of the body, the areas between the volume deflectors 81 said inner layer 113 forms a positive curvature surface protruding into the skin, while the areas in proximity of volume deflectors create a negative curvature forming a void, space, non-contact surface, or channels 1031 (see FIGS. 17, 18 for description of channels). Lines of volume deflectors 81 with spacing 83 parallel to Cross Section B provide not only flexure and bend lines but also the negative curvature used for various functions. Cross Section C is through the thickest portion of a filled containment pack 1. Conformable thermal dressing consists of a set of elongated tubes showing splinting capability and functionality. Long tubes of compressed liquid (filled or in use) created with thermal dressing when wrapped around a limb, additionally provides stabilization along the length of the limb as in a circumferential splint. For example, a trapezoidal shaped containment pack 1 with three linear lines of volume deflectors 81 and spacing 83 perpendicular to the longest dimension of dressing or the dimension that wraps around a limb creates four splints. Four splints forms a basic square that minimally conforms to, form around, or wrap around—a limb. FIG. 11 shows the trapezoidal shaped dressing with eight rectangular splint like features, thus providing more contouring and areas of compression. FIG. 13 is another example of a conformable thermal therapeutic dressing that may be used for acute burns to the face and neck. Volume deflectors 81 are positioned to provide bending or flexure for contour matching portions of the body covered by said containment pack 1. The three dimensional shape of a conformable thermal dressing of FIG. 13 provides maximal treatment coverage for the human face and neck. Clear areas not covered by containment pack 1 are holes 82, 83 for nostrils, mouth, and air exchange. Preloaded component 12 (hereinafter also referred to as preloaded first material 12) is preloaded, preplaced into containment pack 1 in a manufacturing process. Containment pack 1 may be filled or inflated with: a preloaded first material 12; a preloaded first material 12 and at least one rupturable container of introduced component 13 (hereinafter also referred as an introduced second material); or a combination of preloaded first material 12 and introduced second material 13 when the dressing is filled, activated, and/or in use. Volume deflectors 81 are used to form a containment pack with functionality to fit on or around a part of the body, including splinting and pressured sensor coupling. The splint configuration in FIG. 11, said containment pack has at least one volume deflector in more than one line at an angle greater than forty five degrees 84 from the longest dimension of said containment pack and said line spacing 82 is greater than said at least volume deflector spacing 83. For example, when a cooling conformable thermal dressing containment pack 1 is filled with ammonium nitrate and water creating a thermal mass, volume 21 of containment pack 1 forms a set of patterned surfaces as a result of volume deflectors 81. The conformable thermal therapeutic dressing is filled, activated, and wrapped around an injured limb provides therapeutic treatment and stabilization.

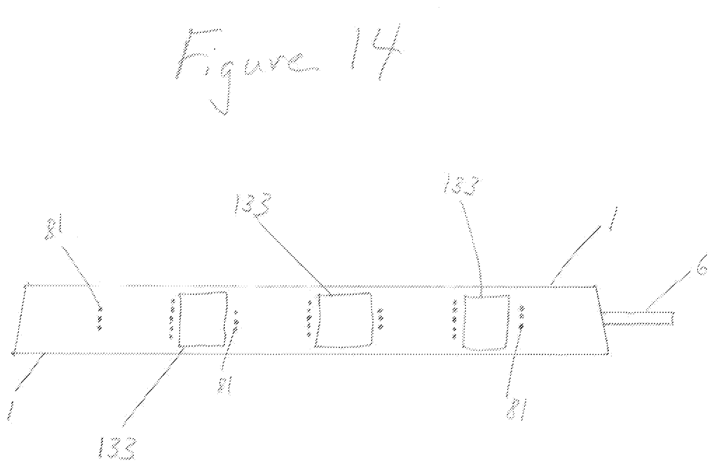

Thermal dressings may have: a port assembly 23 (FIG. 3A) for the introduction of introduced second material 13 (FIG. 3B); or have at least one rupturable container of second material. FIG. 14 shows a particular embodiment, an instant cold pack with volume deflectors 81, a preloaded component 12 of ammonium nitrate and at least one rupturable bag of water 133. Fastener or fastening flap 6 is shown.

FIG. 12 shows the location of sensors 8 (FIG. 10). Sensors 8 are positioned and located on the positive curvature between volume deflectors 81. Sensors 8 are pressed against the skin to provide superior compressive coupling, whereas the areas of volume deflectors 81 between the skin and inner surface 113 are voids, spaces used for wiring, circuitry, and other medicinal interventions. Different types of sensors 8 provide sensed measurements are collected and multiplexed to be used to monitor wounds including temperature, measure movement, and/or oxygen saturation when thermal dressing is in use and over a selected time. Sensing locations are referenced on inner layer and/or may be referenced using a position sensor. Accelerometers like a piezoelectric sensor, also a position sensor, can be used to detect skin motion. Measurement of skin motion would help determine strength of blood flow, the health of the vascular system in proximity to a collection of sensors over or wrapped around, a wound site. Other sensor types include: oxygen saturation measurement using reflection oximetry; and temperature. Sensors are connected to multiplexer 48 (FIG. 10) with sensor circuitry, wired transmission, and/or other transmission medium. Multiplexer 48 may be part of port assembly 23 (FIG. 3A). Multiplexer 48 receives signals from several sensors of at least one sensor type, reduces to a single signal to be transmitted to a collection point as data. Multiplexer 48 may collect signal from multiple sensor types beginning at activation using a recording module, memory bank to record measurements. In a particular embodiment, sensing structure, compressive sensor coupling, a sensing configuration, and sensing measurement collection provides information about the wound and surrounding tissue during the process of applying cooling therapeutic treatment. Three dimensional representations of data sets collected from spatially referenced or located sensors 8 and/or different types of sensors 8 can provide monitoring and diagnostic tools of compromised vascular tissue without removing thermal dressings. Three dimensional graphical representations and/or display information includes; vascular pulsing, skin movement, temperature, and/or oxygen saturation.

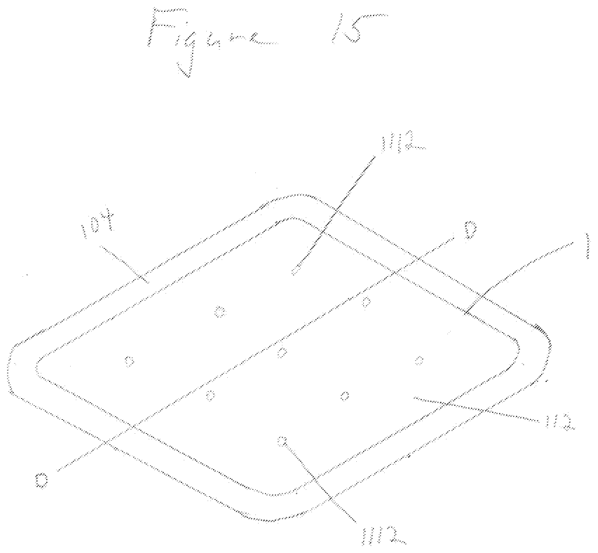

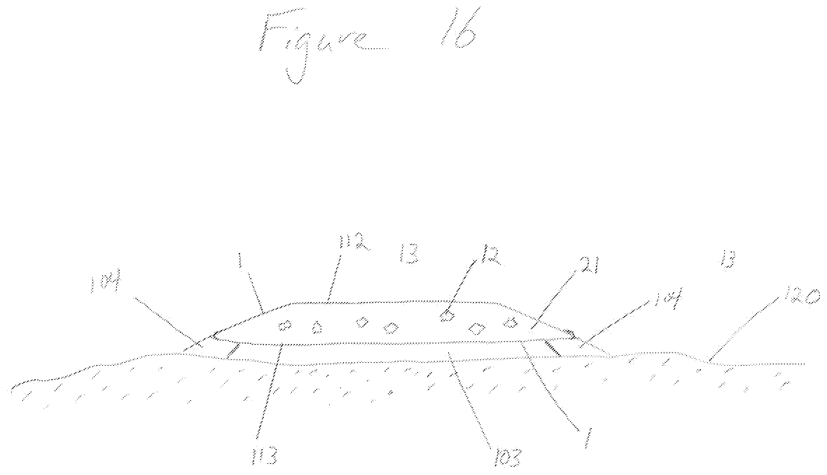

A particular embodiment is thermal dressings with a medical intervention layer 103 including: pharmacological material such as antibiotics, pain relief, vaccines, medicines, and/or biomolecule materials like collagen or keratin; and/or absorptive material for absorbing or wicking fluids from a wound. In the particular embodiment of an exothermic therapeutic dressing, heat provides more effective transdermal infusion of medicines and pharmacological materials. FIG. 15 shows a containment pack 1 with an adhesive layer 104. Adhesive layer 104 peripherally seals dressing to skin. Cross Section D is displayed in FIG. 16. Containment pack 1 has volume 21 and contains a preloaded first material 12. Medical intervention layer 103 is between skin 120 and inner layer 113 of containment pack 1. An exothermic therapeutic bandage or dressing has a preloaded first material 12 may be an exothermic agent such as, an example, granulated iron. Outer layer 112 provides introduction of introduced second material 13 through pores 1112 (FIG. 15). Pores 1112 allow oxygen from the atmosphere, introduced second material 13, to enter the containment pack 1 to activate a chemical thermal reaction providing for an exothermic reaction. Said outer layer 112 may consist of micro pores 1112 in an insulating material. A novel feature is pore density and size can be used or varied to determine the rate of thermal mass. An outer most layer is an oxygen resistant sealing layer to prevent accidental activation, and insures end user removes outer most layer to activate the device at an end user selected time. In another particular embodiment of an endothermic therapeutic dressing: medical intervention layer 103 provides absorbent material to absorb and/or wick away wound fluids such as blood; and provides cooling for damaged tissue. FIG. 17 shows inner layer 113 provides a molded composite surface pattern where the white areas are inner layer 113 and the black intersecting lines are voids or channels 1031 containing absorptive, pharmacological materials and/or biomolecule materials. Volume deflectors 81 are used to create shaping with positive and negative curvatures where negative curvature form voids or channels 1031 to provide medical intervention materials, where molding as in a molded composite layer provides greater, more detailed inner layer 113 patterning. A molded composite surface is a more detailed and precise pattern used for various structures and functional features. Molded composite surface may be a separate layer attached inner layer 113. A special transport pattern for medical intervention layer 103 is shown in FIG. 18 where voids or channels 1031 mimic geomorphic drainage or flow patterns. An example is the wicking of fluids away from wound into a reservoir 105. Special transport pattern may be made using negative curvature patterns of arranged volume deflectors 81. Reservoir 105 may be used for introducing pharmacological intervention into treating site. Molded composite surface or layer may be applied to wound dressings that wrap around, conform, or adhesively attach to part of a body.

What is claimed:

1: A conformable thermal therapeutic dressing comprises:
a containment pack has an inner layer closest to skin and an outer layer furthest from the skin;
said containment pack has at least one volume deflector configured to provide areas of positive and negative curvature when said containment pack is in use;
said at least one volume deflector is positioned to provide at least one contour matching portion of body to be covered by said containment pack; and
said containment pack has a preloaded first material preplaced in a manufacturing process and needed to create a chemical thermal reaction.

2: The conformable thermal therapeutic dressing of claim 1 wherein said containment pack has a plurality of volume deflectors in more than one line at an angle greater than 45 degrees from the longest dimension of said containment pack and said more than one line spacing is greater than spacing between volume deflectors in a line.

3: The conformable thermal therapeutic dressing of claim 1 wherein said containment pack has at least one rupturable pack of introduced second material necessary to create a chemical thermal reaction when mixed with said preloaded first material.

4: The conformable thermal therapeutic dressing of claim 1 wherein said inner layer has absorbent material.

5: The conformable thermal therapeutic dressing of claim 1 wherein said inner layer has pharmacological material.

6: The conformable thermal therapeutic dressing of claim 1 wherein said inner layer has at least one sensor.

7: The conformable thermal therapeutic dressing of claim 1 wherein said inner layer is a molded composite layer.

8: The conformable thermal therapeutic dressing of claim 1 has fasteners to secure dressing to part of the body.

9: A conformable thermal therapeutic dressing with sensors comprises:
a containment pack has an inner layer closest to skin and an outer layer furthest from the skin;
said containment pack has at least one volume deflector configured to provide areas of positive and negative curvature when said containment pack is in use;
said at least one volume deflector is positioned to provide at least one contour matching portions of body to be covered by said containment pack;
said containment pack has a preloaded first material preplaced in a manufacturing process and needed to create a chemical thermal reaction;
said inner layer has at least one located sensor in area of said positive curvature; and
said at least located one sensor is connected to multiplexer with sensor circuitry and wired transmission generally co-located with said negative curvature.

10: The conformable thermal therapeutic dressing with sensors of claim 9 wherein said at least one sensor is spatially located for three dimensional graphical representations of sensed measurements.

11: The conformable thermal therapeutic dressing with sensors of claim 9 wherein said at least one sensor is an accelerometer.

12: The conformable thermal therapeutic dressing with sensors of claim 9 wherein said at least one sensor is a piezoelectric sensor.

13: The conformable thermal therapeutic dressing with sensors of claim 9 wherein said at least one sensor is an electrode, EKG sensor.

14: The conformable thermal therapeutic dressing with sensors of claim 9 wherein said at least one sensor is a photoplethysmography sensor for reflection oximetry.

15: The conformable thermal therapeutic dressing with sensors of claim 9 wherein said at least one sensor is at least one sensor type.

16: A thermal therapeutic dressing with pharmacological material comprising:
a containment pack has an inner layer closest to skin and an outer layer furthest from the skin;
said inner layer has pharmacological material;
said containment pack has a preloaded first material preplaced in a manufacturing process and needed to create a chemical thermal reaction; and
said outer surface has opening configured to accept an introduced second material.

17: The thermal therapeutic dressing with pharmacological material of claim 16 has an adhesive material configured to attach dressing to a part of the body.

18: The thermal therapeutic dressing with pharmacological material of claim 16 wherein said outer layer is an insulating material with pores.

19: The thermal therapeutic dressing with pharmacological material of claim 16 has a reservoir.

20: The thermal therapeutic dressing with pharmacological material of claim 16 wherein said layer is a molded composite layer of direct thermal treatment and a pharmacological material.
